(19) United States
(12) Patent Application Publication
(10) Pub. No.: US 2006/0003001 A1
(43) Pub. Date: Jan. 5, 2006

(54) Chronotherapeutic compositions and methods of their use

(76) Inventors: John Devane, Athlone (IE); Jackie Butler, Athlone (IE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/053,865

(22) Filed: Feb. 10, 2005

Related U.S. Application Data
(60) Provisional application No. 60/543,402, filed on Feb. 11, 2004.

(51) Int. Cl. A61K 31/137; A61K 9/22

(52) U.S. Cl. 424/468; 514/651

(57) ABSTRACT

Chronotherapeutic formulations of cardiovascular drugs are disclosed. The formulations comprise at least one cardiovascular drug that exhibits an in vivo elimination half-life of less than about 8 hours; wherein the formulation exhibits the following in vivo profile following administration to a subject: a) a delay in release of therapeutic levels of the at least one drug for about 2 to about 8 hours;
b) a Tₘₐₓ at about 8 to about 12 hours; c) a drug plasma level within 50% of the peak for greater than or equal to 12 hours; and d) a peak-to-trough ratio of drug plasma levels greater than or equal to about 4.

This application claims the benefit of priority of U.S. Provisional Application No. 60/543,402, filed Feb. 11, 2004, which is incorporated by reference herein.

Chronotherapy involves the synchronization of drug exposure with the circadian pattern of disease symptoms or underlying physiological functions. Such therapies provide a more rational or targeted approach for treating a disease. For example, many cardiovascular diseases present well-established circadian patterns that include early morning surges in blood pressure, heart rate, cardiac contractility, coronary blood vessel tone, and other functions. A chronotherapeutic formulation can target optimal drug exposure to the early morning period (e.g., about 6 AM to about 10 AM) during a course of treatment.

In addition, chronotherapeutic formulations improve patient compliance by permitting a once-daily night time administration that delays the release of the drug until it is needed during the early morning period, while maintaining therapeutic concentrations during waking hours. Such once-daily formulations are desirable because patient compliance can be as high as 80%, while with twice-a-day and three times-a-day dosing, compliance levels fall to 60% and 40%, respectively. See, e.g., Shilo, et al., Ann. Pharmacother., 35 (11):1339-42, 2001. Thus, chronotherapeutic dosage forms that reduce the frequency of administration can significantly improve the therapeutic outcome.

Some chronotherapeutic formulations of cardiovascular drugs have been described. See, e.g., WO 02/072034, U.S. Pat. Nos. 5,788,987; 5,891,474, 6,190,692; 6,500,454, and 6,620,439; U.S. Patent Application 20030082230, published May 1, 2003; and U.S. Patent Application 20030190360, published Oct. 3, 2003. These formulations have been designed to create a delay, or lag, in initial drug release that reportedly synchronizes the onset of drug absorption and exposure with the early morning risk period. Such formulations have typically been described as having a lag time of between 2 to 8 hours following a single dose of the administration.

For example, Busetti (U.S. Pat. Nos. 5,788,987; 5,891,474; 6,190,692) describes a delayed-release formulation that, when administered prior to sleep, produces a therapeutically effective concentration of an active compound at about the time of awakening. The formulation is prepared by coating a drug core with a swellable polymer; the length of the delay in release of the drug depends on the thickness of the polymeric coating. After the delay period, during which the polymeric coating is removed by dissolution or erosion, the active compound is exposed and rapidly released into the subject's system.

Busetti does not describe a dosage form that achieves a delayed and extended release of an active compound, providing a therapeutic benefit beyond the early morning hours and throughout the day. Instead, this type of rapid release provides an initial spike (i.e., a burst release) followed by a rapid decline in the plasma concentration of the drug. Thus, while drug may be present at a therapeutic level during the early morning hours (i.e., during the initial spike), that level is not maintained throughout the waking hours of the day. Consequently, this approach to therapy does not provide a subject with adequate or optimal protection throughout the day.

Moreover, the singular focus on lag times overlooks many other important parameters that impact the efficacy of a chronotherapeutic formulation. For example, a drug having a long elimination half-life may be formulated with a standard lag phase and also provide adequate coverage throughout the day but may accumulate with repeated doses. In contrast, a drug having a short elimination half-life will not achieve sustained therapeutic blood levels if it is formulated simply with a standard lag phase because it is cleared much more quickly from the subject's system. Thus, in the case of short elimination half-life drugs, additional parameters must be addressed to prepare suitable chronotherapeutic formulations. Such parameters include the drug absorption rate, the timing of peak concentrations, the duration of therapeutic blood levels, the elimination half-life of the drug and the duration of the washout of blood levels necessary to achieve an optimal chronotherapeutic plasma profile suitable for repeated dosing. The present invention provides formulations suitable for use with short elimination half-life cardiovascular drugs.

One important class of cardiovascular drugs is beta-blockers. Beta-blockers are beta-adrenoceptor selective antagonists, and include well-known commercial products such as propanolol and atenolol. The drugs act by blocking neurotransmitter action at beta-adrenergic receptors and, as a consequence, disrupt transmission in the sympathetic nervous system. The effects of blocking beta-adrenergic receptors are widespread, reflecting the distribution of these receptors throughout the body. They include, but are not limited to, effects on the heart and cardiovascular system, the gastrointestinal tract, the respiratory tract, the eye, the liver, and the genitourinary system. These effects and others are described, for example, in textbooks such as Goodman and Gilman's The Pharmacological Basis of Therapeutics (McGraw Hill, 1996) and Rang, Dale and Ritter's Pharmacology (Churchill Livingstone, 1999).

Beta-blockers are indicated for the treatment of a number of conditions including, but not limited to, hypertension, ischemic heart disease, atrial fibrillation, congestive heart failure, peripheral arterial occlusive disease, angina pectoris, cardiac arrhythmias, heart failure, glaucoma, migraine, the effects of thyroid disease, and symptoms of anxiety, such as palpitations. They are most commonly used in diseases of the cardiovascular system.

A general mechanism of action for beta-blockers on the cardiovascular system has been elucidated. In both vascular and cardiac tissue, muscle cell contraction occurs when cells are stimulated by catecholamines binding to adrenergic receptors. This can lead to increases in heart rate, blood pressure, and in the velocity and force of myocardial contraction, among other things. Beta-blockers antagonize certain of these effects of catecholamines, resulting in vasodilation, reduced blood pressure, and a reduction in the force required to pump blood from the heart.

Metoprolol (1-(isopropylamino)-3-[p-(2-methoxyethyl)phenoxy]-2-propanol) is one beta-blocker that is typically prescribed for hypertension, angina pectoris, and stable or symptomatic heart failure. The compound preferentially acts on beta-1-adrenoreceptors, which predominate in cardiac muscle. Thus, the drug is relatively selective for cardiac tissues. However, at higher concentrations, this selectivity is diminished as the drug also blocks beta-2-adrenoceptors in other parts of the body (e.g., in vascular and bronchial tissues).

Like many cardiovascular drugs, several of the beta-blockers are limited in their effectiveness as chronotherapeutics because they exhibit a short elimination half-life in a patient following administration. For example, metoprolol has a relatively short elimination half-life of about 3.5 hours. As a result of the short elimination half-life, subjects taking drugs like metoprolol require multiple daily doses to ensure continuous protection. This generates significant problems with subject compliance and maintenance of therapeutic levels in the subject's system throughout the day.

In addition, sharp peaks and drops in the plasma concentration of short elimination half-life drugs (caused by multiple daily administrations) result in undesirable side-effects. As noted above, for example, the selectivity of metoprolol for beta-1-adrenoreceptors decreases at higher plasma concentrations. Thus, unwanted effects are observed in non-cardiac tissues when the plasma concentration of the drug is too high.

Certain sustained-release formulations of cardiovascular drugs have been designed for once-a-day administration. For example, conventional sustained-release formulations of metoprolol reportedly provide a continuous therapeutic plasma level of metoprolol for at least 24-hours. See, e.g., Plosker, et al., “Controlled Release Metoprolol Formulations,” Drugs 43 (3): 382-414, 1992; Kendall, et al., “Controlled Release Metoprolol,” Clin. Pharmacokinet, 21 (5): 319-330,1991; U.S. Pat. No. 4,036,227; U.S. Pat. No. 4,792,452; U.S. Pat. No. 4,871,549; U.S. Pat. No. 4,927,640; U.S. Pat. No. 4,957,745; U.S. Pat. No. 5,081,154; U.S. Pat. No. 5,169,638; and U.S. Pat. No. 5,399,362.

These once-daily dosage forms reportedly achieve continuous 24-hour therapy by quickly raising the subject's drug plasma level above a therapeutic threshold, and keeping it there through a full 24 hour period. This blanket 24-hour coverage, however, is not the most effective or desirable form of chronotherapy. For example, by delivering constant amounts of the drug day after day, the drug plasma profile shifts from one administration to the next and is not reproducible. In other words, the plasma levels observed during the first administration differ from those observed in subsequent administrations of the drug, because not all of the drug clears the subject's system before the next dose is taken. Consequently, the kinetic parameters (time of coverage, peak-to-trough ratios, timing of lag and washout phases, etc.) are distorted over a course of repeated dosing. This adds a layer of unpredictability and complexity to any treatment protocol that is difficult for a clinician to accurately account for.

In addition, as with many cardiovascular drugs, long-term continuous administration often results in tolerance or desensitization to the drug. As a result, ever increasing amounts of the drug must be administered to maintain therapeutic efficacy. Unfortunately, the amounts of drug that may be administered are often dose-limited by adverse side-effects caused by the drug. Thus, the development of desensitization in a subject can ultimately eliminate important long-term therapeutic options for treating a particular cardiovascular condition with drugs.

This long-term desensitization, of course, differs from the acute tolerance associated with cardiovascular nitrate drugs. Acute tolerance can be observed in a patient after a single administration of a nitrate drug. Accordingly, there is a rapid loss or reduction in the responsiveness of the target tissue to a nitrate therapy. The effects of acute nitrate tolerance are well-known in the art and have been addressed by a number of formulations suited to combat this unique problem. For example, pending U.S. application Ser. No. 10/214,345 describes an oral once-daily chronotherapeutic nitrate formulation that provides a lag time prior to release, and a combination of therapeutic/non-therapeutic exposure periods to minimize acute nitrate tolerance. These formulations, however, are specifically designed to avoid acute nitrate tolerance and are unique to the field of nitrate therapy. Moreover, the formulations are defined only in terms of therapeutic/non-therapeutic plasma nitrate concentrations (i.e., above or below 100 ng/ml). Consequently, the approaches to overcoming acute nitrate tolerance are not generally applicable to avoiding the long-term desensitization associated with non-nitrate therapies.

In addition to problems with long-term desensitization, constant exposure to many cardiovascular drugs presents complications when the therapy is suddenly discontinued. This may occur, for example, when a subject does not have access to his or her medication, or when the drug administration must be halted for medical reasons (e.g., due to side-effects, negative interactions with other medications, surgical complications, etc.).

When beta-blocker therapy is discontinued following a course of continuous treatment, subjects experience a “rebound phenomenon.” In one study, subjects developed untoward ischemic events and serious withdrawal complications, including intermediate coronary syndrome, ventricular tachycardia, fatal myocardial infarction, and sudden death, within two weeks of suddenly discontinuing their beta-blocker therapy. See, e.g., RR Miller, et al., “Propranolol-withdrawal rebound phenomenon. Exacerbation of coronary events after abrupt cessation of antianginal therapy,” New England Journal of Medicine, 293:416-418 (1975). The package insert for one commercially available extended release form of metoprolol also warns that angina pectoris is exacerbated, and in some cases, myocardial infarction has occurred, following abrupt cessation of treatment. See Package Drug Insert, TOPROL-XL™ (metoprolol succinate) (Rev. 11/2002).

Consequently, beta-blockers must be gradually reduced following a course of chronic administration, and activity must be restricted during the withdrawal period. This caution, however, does not account for situations where cessation of treatment cannot be avoided (e.g., when a patient unexpectedly does not have access to the medication). Thus, the danger of “rebound” caused by long-term exposure to cardiovascular drugs remains a significant therapeutic concern.

Finally, as with most drugs, subjects experience undesirable side-effects from continuous exposure to the drug. In the case of beta-blockers, such as metoprolol, the side-effects are well-documented and include headaches and dizziness, depression, memory loss, insomnia, nausea, diarrhea and other gastrointestinal disorders, and shortness of breath, among other things. Many of these side-effects are transitory, but continuous 24-hour exposure to the drug provides opportunities for repeated adverse events in susceptible subjects.

Given these various therapeutic challenges, simply providing a lag in release followed by continuous 24-hour exposure to a drug should not be the only goal of an effective chronotherapeutic drug therapy. The optimal formulation should do much more. For example, a more safe and effective approach should tailor the extended drug release to provide appropriate coverage during the periods when it is most needed, limit unnecessary fluctuations in drug levels, and allow for beneficial drug-free intervals when therapy is not needed. In so doing, a clinically efficacious, reproducible daily drug release profile is achieved while preventing, treating, and/or managing cardiovascular conditions. Such a therapy may also prevent or reduce side-effects, including any rebound phenomenon or tolerance. There is a need in the art for new effective drug formulations of this type.

The present invention provides formulations of cardiovascular drugs that achieve a specific therapeutic blood level profile, while avoiding limitations associated with prior formulations. The formulations of the invention are particularly suitable for use as once-daily chronotherapeutic formulations. Thus, in some embodiments, the formulations may be administered at night while providing therapeutic coverage during the early morning hours and throughout the following day. Moreover, the present formulations achieve a blood level profile that is reproducible following subsequent administrations of the drug.

### BRIEF DESCRIPTION OF THE FIGURES

FIG. 1 illustrates the simulated relationship at steady-state between lag-times and absorption half-lives for drugs with different elimination half-lives.

FIG. 2 illustrates simulated steady-state data for a metropolol tartrate formulation with a four-hour lag-time and a range of absorption half-lives.

### DEFINITIONS

As used herein, the term “absorption half-life” refers to the time required for 50% of a drug to be absorbed following administration to a subject.

As used herein, the terms “beta-blocker” and “beta adrenergic blocker” refer to the class of compounds that generally block the binding of agonists to β-adrenoceptors. Beta-blockers are typically used for preventing, treating, and/or managing a range of ailments, such as hypertension, angina pectoris, myocardial infarction, cardiac arrhythmia, migraines, tremors, anxiety, and glaucoma. Beta-blockers include oxyprenolol, pindolol, acebutolol, celiprolol, atenolol, nadolol, sotalol, labetalol, carvedilol, nevibolol, betaxolol, bisoprolol, metoprolol, timolol, propranolol, and esmolol. The term also includes all forms of beta-blockers, including racemates, stereoisomers, and any pharmaceutically acceptable salts thereof. In one embodiment, the beta-blocker is metoprolol.

As used herein, the term “cardiovascular condition” refers to diseases of the cardiovascular system, and symptoms thereof. Cardiovascular conditions are known in the art and include, but are not limited to, hypertension, angina, coronary artery disease, cerebrovascular disease, peripheral vascular disease, myocardial infarction, stroke, and thrombosis.

As used herein, the term “cardiovascular drug” refers to drug compounds and/or formulations that are suitable for treating, preventing, and/or managing cardiovascular conditions in a subject. Such drugs include, but are not limited to, peripheral alpha or beta adrenergic blockers, central alpha or beta adrenergic blockers, mixed alpha/beta adrenergic blockers, angiotensin converting enzymes (ACE) inhibitors, angiotensin II receptor antagonists, antiarrhythmics (groups I, II, and III), calcium channel blockers, potassium channel activators (e.g., Nicorandil), aldosterone antagonists, renin inhibitors, diuretics, and vasodilators (coronary, peripheral, and pulmonary). In a particular embodiment, the cardiovascular drug is a beta adrenergic blocker, calcium antagonist, a potassium channel activator (e.g., Nicorandil), or ACE inhibitor. The term includes all forms of such drugs, including stereoisomers and any pharmaceutically acceptable salts thereof. The invention encompasses formulations that provide a combination of cardiovascular drugs.

As used herein, the phrase “delayed release formulation” refers to a pharmaceutical preparation that substantially or completely withholds or impairs delivery of a compound for a specified period of time, i.e., the delay period. Following this delay period, the active ingredient of such formulations begins to be released. Without further impairment, the full amount of the drug is released rapidly. For example, a typical delayed-release tablet will inhibit release of its active compound until an exterior coating disintegrates or erodes. Once the coating is dissolved, the active compound is rapidly released into the subject.

As used herein, the term “elimination half-life” refers to the time required for 50% of a drug to be eliminated following administration to a subject. A “short elimination half-life drug” is one that exhibits an elimination half-life (t½) of less than 8 hours following administration to a subject. Examples of drugs having a short elimination half-life are provided in Table 1. One of skill in the art is familiar with the half-life of any given drug and methods for determining the same. For example, the elimination half-life of a drug is typically estimated as [ln2/keI], where keI=[(InC1−InC2)/(t2−t1)]. C1 and C2 are concentrations at time t1 and t2, respectively, in the log-linear terminal phase of the plasma concentration versus time curve.

TABLE 1

| | Elimination |
|---|---|
| Drug | Half-Life (h) |
| Acebutolol | 2.7 |
| n-Acetylprocainamide | 6.0 |
| Acetylsalicylic acid | 0.25 |
| Alprenolol | 2.5 |
| Carvedilol (i/v) | 2.4 |
| Carvedilol (po) | 6.4 |
| Oxprenolol | 2.5 |
| Hydralazine | 1.0 |
| Isradipine | 3.8 |
| Prazosin | 2.9 |
| Atenolol | 6.1 |
| Captopril | 2.2 |
| Chlorothiazide | 1.5 |
| Diltiazem | 3.7 |
| Disopyramide | 6.0 |
| Furosemide | 1.5 |
| Hydrochlorothiazide | 2.5 |
| Labetalol | 4.9 |
| Methyldopa | 1.8 |
| Metoprolol | 3.5 |
| Nicardipine | 1.3 |
| Nicorandil | 1.1 |
| Nifedipine | 1.8 |
| Pindolol | 3.6 |
| Procainamide | 3.0 |
| Propranolol | 3.9 |
| Quinidine | 6.2 |
| Spironolactone | 1.6 |
| Timolol | 4.1 |
| Verapamil | 4.0 |

The term “pharmaceutically acceptable salt” includes salts that are physiologically tolerated by a subject. Such salts are typically prepared from an inorganic and/or organic acid. Examples of suitable inorganic acids include, but are not limited to, hydrochloric, hydrobromic, hydroiodic, nitric, sulfuric, and phosphoric acid. Organic acids may be aliphatic, aromatic, carboxylic, and/or sulfonic acids. Suitable organic acids include, but are not limited to, formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic (besylate), stearic, sulfanilic, alginic, galacturonic, and the like.

As noted above, in some embodiments metoprolol is the beta-blocker used in the present invention. The particular metoprolol salt may be selected on the basis of its solubility, as needed to achieve the desired pharmaceutical and/or pharmacokinetic properties in the formulation. Examples of very soluble salts include the tartrate and hydrochloride salts. In one embodiment, the beta-blocker is a tartrate salt of metoprolol. Solubility considerations may also be used to select particular salts from among the other cardiovascular drugs encompassed by the present invention.

As used herein, the term “pharmaceutically acceptable excipient” includes compounds that are compatible with the other ingredients in a pharmaceutical formulation and not injurious to the subject when administered in acceptable amounts.

As used herein, the phrase “therapeutically effective amount” refers to the amount of a drug compound, or pharmaceutically acceptable salt thereof, that alone and/or in combination with other drugs provides a benefit in preventing, treating, and/or managing one or more conditions that may benefit from the properties of that particular drug.

As used herein, the phrase “extended release formulation” or “extended release dosage form” refers to a pharmaceutical preparation that maintains a therapeutically effective level of an active compound in a subject for a specified period of time. An extended release formulation may be designed to delay the release of the active compound for a specified period of time. Such compounds are referred to herein as “delayed onset, extended release formulations” or “delayed onset, extended release dosage forms.”

The term “Tₘₐₓ” refers to the time at which the peak level of drug plasma level is attained in a subject following administration of the drug to the subject.

The term “lag-time” refers to the time before the first quantifiable plasma concentration in the plasma concentration versus time curve.

The terms “peak-to-trough fluctuation” or “peak-to-trough ratio” refer to the ratio of the peak plasma concentration to the minimum plasma concentration in a dosing interval at steady-state.

The term “time cover” refers to the duration of time in a dosing interval at steady-state that plasma concentrations are above a minimum concentration defined in this application as 50% of the peak concentration.

### DESCRIPTION OF THE INVENTION

The present invention is directed to compositions and methods for preventing, treating, and/or managing conditions that are preventable, treatable, and/or manageable with cardiovascular drugs. The invention is particularly suitable for cardiovascular drugs that exhibit a short elimination half-life following administration to a subject.

In one embodiment, the present invention relates to delayed onset, extended release formulations comprising one or more short elimination half-life cardiovascular drugs, and methods of their use in preventing, treating, and/or managing cardiovascular conditions. In some embodiments, the present invention relates to delayed onset, extended release formulations comprising one or more short elimination half-life cardiovascular drugs, and methods of their use, in providing an effective therapy for such conditions while maintaining a reproducible daily drug release profile. In further embodiments, the present invention relates to delayed onset, extended release formulations comprising one or more short elimination half-life cardiovascular drugs, and methods of their use, in providing an effective therapy for such conditions while preventing and/or reducing side-effects, rebound phenomenon, tolerance and/or desensitization.

In some embodiments, the invention relates to delayed onset, extended release formulations comprising one or more beta-blockers, and methods of their use in preventing, treating, and/or managing cardiovascular conditions. In some embodiments, the present invention relates to delayed onset, extended release formulations comprising one or more beta-blockers, and methods of their use, in providing an effective therapy for such conditions while maintaining a reproducible daily drug release profile. In further embodiments, the present invention relates to delayed onset, extended release formulations comprising one or more beta-blockers, and methods of their use, in providing an effective therapy for such conditions while preventing and/or reducing side-effects, rebound phenomenon, tolerance and/or desensitization.

The present formulations overcome deficiencies associated with prior art formulations of cardiovascular drugs. In particular, the present formulations avoid or reduce long-term desensitization, rebound phenomena, and various undesirable side effects, while maintaining a reliable and reproducible drug plasma profile that is consistent over a course of multiple doses.

The present formulations are suitable for use as chronotherapeutics for once-daily administration. In some embodiments, the chronotherapeutic formulation is administered at night, with release of the short elimination half-life cardiovascular drug delayed until the early morning hours. Formulations of the present invention are defined as those exhibiting the following in vivo chronotherapeutic profile following administration to a subject:
1) a delay in release of about 2 to about 8 hours, providing therapeutic levels of drug during the early morning “high-risk” period when administered at night;
2) a Tₘₐₓ at about 8 to about 12 hours, such that, when administered at night, peak drug levels coincide with periods of time when the therapeutic levels of the drug are most needed by the subject receiving the administration;
3) a plateau drug plasma level within 50% of the peak for greater than or equal to 12 hours, to provide adequate therapeutic drug coverage, when administered at night, throughout the active phases of the day (e.g., 6 AM until bedtime); and
4) a peak-to-trough ratio of drug plasma levels greater than or equal to about 4, so that sub-therapeutic levels occur at some point during the dosing period.

The present formulations are designed to satisfy these parameters, while taking into account the varying absorption half-life and elimination half-life values of different cardiovascular drugs. In particular, the present invention is suitable for using short elimination half-life cardiovascular drugs in chronotherapeutic formulations.

The delay in the release of therapeutic concentrations of the short elimination half-life cardiovascular drug(s) may be from about 2 to about 8 hours, from about 3 to about 8 hours, or from about 3 to about 6 hours, or any hour or fraction of time in between, following administration of the formulation. For example, the present controlled-release formulations may delay release of therapeutic concentrations of the short elimination half-life cardiovascular drug(s) for about 2, 3, 4, 5, 6, 7, or 8 hours, or any hour or fraction of time in between, following administration.

Following release of the drug, therapeutic levels of the drug may be maintained for at least 12 hours. Typically, the short elimination half-life cardiovascular drug(s) is maintained at or above the therapeutic level for about 12 to about 20 hours, or any hour or fraction of time in between, measured from the time of administration. Accordingly, the cardiovascular drug(s) is maintained at or above the therapeutic level for about 12, 13, 14, 14, 16, 17, 18, 19 or 20 hours, or any hour or fraction of time in between, measured from the time of administration. In this manner, the present formulations provide therapeutically effective amounts of the drug throughout the day.

The formulations also provide for a “washout phase” by requiring a peak-to-trough ratio of greater than or equal to about 4. As compared to the maximum cardiovascular drug plasma levels attained following release of the drug, the level to which the blood plasma concentration falls during a washout period exhibits a ratio (peak-to-trough) of greater than about 4:1. Thus, the peak-to-trough ratio may be about 4:1, 5:1, 6:1, 7:1, 8:1, 9:1, 10:1, or greater, or any fraction in between.

In so doing, the plasma concentration of the short elimination half-life cardiovascular drug(s) in the blood stream of the subject is allowed to drop below the minimum therapeutic level until the next dose of the drug is administered. In some particular formulations, a washout phase may be provided by the delay phase of a subsequent dosage form. In other words, the plasma levels of short elimination half-life cardiovascular drug(s) in the blood stream of the subject following a first administration are allowed to drop below the minimum therapeutic level and remain there during the delay phase of a subsequently administered dose. A typical washout phase will last from about 1 or less hours to about 8 hours, or any hour or fraction of time in between. Thus, the washout phase may last 0.5, 1, 2, 3, 4, 5, 6, 7, or 8 hours, or any hour or fraction of time in between.

The therapeutically effective level for the short elimination half-life cardiovascular drug(s) may vary depending on the drug being used, the patient, and the condition being treated. In some instances, the therapeutically effective level may be determined empirically by determining a subject's response and titrating a dose as necessary. Such experimentation is routine and within the skill in the art. In one embodiment, where metoprolol is provided in the formulation, the daily dose is about 1 mg to about 600 mg, or any number in between, for example, about 12.5 mg to about 400 mg.

By administering the present formulations, a subject receiving treatment can avoid or reduce the effects associated with the withdrawal from the drug (i.e., rebound phenomenon). Likewise, an individual who is already taking a cardiovascular drug formulation may substitute or switch to one of the presently disclosed formulations in order to receive the same benefit. In cases where the subject must intentionally be withdrawn from a cardiovascular drug formulation, but desires to avoid the rebound phenomenon, it is advantageous for the subject to switch to one of the presently disclosed formulations for at least about 7 days before ceasing treatment. This will provide adequate time for the subject to adjust before withdrawal from the drug is permitted.

The methods of the present invention involve administering a pharmaceutically effective amount of at least one short elimination half-life cardiovascular drug, or a pharmaceutically acceptable salt thereof, to a subject in need of such treatment. Suitable short elimination half-life cardiovascular drugs are described above. In some embodiments, the short elimination half-life cardiovascular drug is a beta-blocker, calcium antagonist, or ACE inhibitor. In a particular embodiment, the cardiovascular drug may be metoprolol.

The cardiovascular conditions that may be prevented, treated, and/or managed using the inventive compositions and methods include, but are not limited to, hypertension, angina, coronary artery disease, cerebrovascular disease, peripheral vascular disease, myocardial infarction, stroke, and thrombosis. In some embodiments, the conditions being treated, prevented, or managed include hypertension, angina, or myocardial infarction. Other conditions and symptoms of cardiovascular conditions that involve abnormal cardiovascular activity may also be treated, prevented, or managed using the presently disclosed formulations and methods.

At least one short elimination half-life cardiovascular drug, or a pharmaceutically acceptable salt thereof, may be provided in a pharmaceutical composition for use according to the present invention. Such compositions optionally include one or more pharmaceutically acceptable excipients. Suitable excipients are known to those of skill in the art and are described, for example, in the Handbook of Pharmaceutical Excipients (Kibbe (ed.), 3^{rd} Edition (2000), American Pharmaceutical Association, Washington, D.C.), and Remington's Pharmaceutical Sciences (Gennaro (ed.), 20^{th} edition (2000), Mack Publishing, Inc., Easton, Pa.), which, for their disclosures relating to excipients and dosage forms, are incorporated herein by reference.

Suitable excipients include, but are not limited to, starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, plasticizers, gelling agents, thickeners, hardeners, setting agents, suspending agents, surfactants, humectants, carriers, stabilizers, antioxidants, and combinations thereof.

The pharmaceutical compositions of the invention are typically provided in dosage forms that are suitable for administration to a subject by a desired route. A number of suitable dosage forms are described below, but are not meant to include all possible choices. One of skill in the art is familiar with the various dosage forms that are suitable for use in the present invention, as described, for example, in Remington's Pharmaceutical Sciences, portions of which have been incorporated by reference above. The most suitable route in any given case will depend on the nature and severity of the condition being prevented, treated, and/or managed. The pharmaceutical compositions of this invention may be formulated for administration orally, nasally, rectally, intravaginally, intracisternally, and topically (including buccally and sublingually).

Formulations suitable for oral administration include, but are not limited to, capsules, cachets, pills, tablets, lozenges (which may use a flavored base, usually sucrose and acacia or tragacanth), powders, granules, solutions, suspensions in an aqueous or non-aqueous liquid, oil-in-water or water-in-oil liquid emulsions, elixirs, syrups, pastilles (which may use an inert base, such as gelatin and glycerin, or sucrose and acacia), pastes, and the like.

In solid dosage forms for oral administration (capsules, tablets, pills, powders, granules, and the like), suitable excipients include, but are not limited to, carriers, such as sodium citrate or dicalcium phosphate; fillers or extenders, such as starches, lactose, sucrose, glucose, mannitol, or silicic acid; binders, such as hydroxymethyl-cellulose, alginates, gelatin, polyvinylpyrrolidone, sucrose or acacia; humectants, such as glycerol; disintegrating agents, such as agar, calcium carbonate, potato or tapioca starch, alginic acid, certain silicates, or sodium carbonate; solution retarding agents, such as paraffin; absorption accelerators, such as quaternary ammonium compounds; wetting agents, such as cetyl alcohol or glycerol monostearate; absorbents, such as kaolin and bentonite clay; lubricants, such as talc, calcium stearate, magnesium stearate, solid polyethylene glycols, and sodium lauryl sulfate; coloring agents; buffering agents; dispersing agents; preservatives; and diluents. The aforementioned excipients are given as examples only and are not meant to include all possible choices. Solid compositions may also be employed as fillers in soft and hard-filled gelatin capsules using excipients such as lactose or milk sugars, high molecular weight polyethylene glycols, and the like. Any of these dosage forms may optionally be scored or prepared with coatings and shells, such as enteric coatings and coatings for modifying the rate of release, examples of which are well known in the pharmaceutical-formulating art.

Suitable liquid dosage forms for oral administration include emulsions, microemulsions, suspensions, syrups, and elixirs. These formulations may optionally include diluents commonly used in the art, such as, for example, water or other solvents, solubilizing agents and emulsifiers, including, but not limited to, ethyl alcohol, isopropyl alcohol, ethyl carbonate, ethyl acetate, benzyl alcohol, benzyl benzoate, propylene glycol, 1,3-butylene glycol, oils, glycerol, tetrahydrofurfuryl alcohol, polyethylene glycols, fatty acid esters of sorbitan, and mixtures thereof. In addition, the liquid formulations optionally include adjuvants such as wetting agents, emulsifying and suspending agents, sweetening, flavoring, coloring, perfuming, and preservative agents. Suitable suspension agents include, but are not limited to, ethoxylated isostearyl alcohols, polyoxyethylene sorbitol and sorbitan esters, microcrystalline cellulose, aluminum metahydroxide, bentonite, agar-agar and tragacanth, and mixtures thereof. Liquids may be delivered as-is, or in a carrier, such as a hard or soft capsule or the like.

For rectal or vaginal administration, the composition may be provided as a suppository. Suppositories optionally include one or more non-irritating excipients, for example, polyethylene glycol, a suppository wax, or a salicylate. Such excipients may be selected based on desirable physical properties. For example, a compound that is solid at room temperature but liquid at body temperature will melt in the rectum or vaginal cavity and release the active compound. The formulation may alternatively be provided as an enema for rectal delivery. Formulations suitable for vaginal administration also include pessaries, tampons, creams, gels, pastes, foams, or spray formulations containing such carriers, examples of which are known in the art.

Formulations suitable for topical or transdermal administration include powders, sprays, ointments, pastes, creams, lotions, gels, solutions, patches, and inhalants. Such formulations optionally contain excipients such as animal and vegetable fats, oils, waxes, paraffins, starch, tragacanth, cellulose derivatives, polyethylene glycols, silicones, bentonites, silicic acid, talc, zinc oxide, or mixtures thereof. Powders and sprays may also contain excipients such as lactose, talc, silicic acid, aluminum hydroxide, calcium silicates, and polyamide powder. Additionally, sprays may contain propellants, such as chlorofluoro-hydrocarbons and volatile unsubstituted hydrocarbons, such as butane and propane.

Transdermal patches have the added advantage of providing controlled delivery of the drug into the subject's body. Such dosage forms can be made by dissolving, dispersing, or otherwise incorporating a pharmaceutical composition containing at least one cardiovascular drug in a suitable medium, such as an elastomeric matrix material. Absorption enhancers can also be used to increase the flux of the mixture across the skin. The rate of such flux may be controlled by providing a rate-controlling membrane or dispersing the compound in a polymer matrix or gel.

For parenteral administration, such as administration by injection (including, but not limited to, subcutaneous, bolus injection, intramuscular, intraperitoneal, and intravenous), the pharmaceutical compositions may be formulated as isotonic suspensions, solutions, or emulsions, in oily or aqueous vehicles, and may contain formulatory agents such as suspending, stabilizing, or dispersing agents. Alternatively, the compositions may be provided in dry form such as a powder, crystalline, or freeze-dried solid, for reconstitution with sterile pyrogen-free water or isotonic saline before use. They may be presented, for example, in sterile ampoules or vials.

Examples of suitable aqueous and nonaqueous excipients include water, ethanol, polyols (such as glycerol, propylene glycol, polyethylene glycol, and the like), oils, injectable organic esters, and mixtures thereof. Proper fluidity can be maintained, for example, by the use of surfactants.

These compositions may also contain adjuvants such as preservatives, wetting agents, emulsifying agents, and dispersing agents. Preventing the action of microorganisms may be achieved by including various antibacterial and/or antifungal agents, for example, paraben, chlorobutanol, phenol sorbic acid, and the like. It may also be desirable to include isotonic agents, such as sugars, sodium chloride, and the like in the compositions.

To prolong the therapeutic effect of a drug, it may be desirable to slow the absorption of the drug from a subcutaneous or intramuscular injection. Prolonged absorption of the injectable pharmaceutical form may be brought about by the inclusion of agents that delay absorption, such as aluminum monostearate and/or gelatin. This may also be accomplished by the use of a liquid suspension of crystalline or amorphous material having low solubility. The rate of absorption of the drug then generally depends upon its rate of dissolution, which may depend upon crystal size and crystalline form. Alternatively, delayed absorption of a parenterally-administered form can be accomplished by dissolving or suspending the drug in an oil vehicle.

In addition to the common dosage forms discussed above, the pharmaceutical compositions may also be administered by controlled-release delivery devices, examples of which are well known to those of ordinary skill in the art. Examples of different formulations are provided in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, the disclosures of which, for their discussions of pharmaceutical formulations, are incorporated herein by reference. Advantages of controlled-release formulations may include extended activity of the drug, reduced dosage frequency, decreased side-effects (including rebound phenomena, desensitization, and tolerance), and increased patient compliance. Suitable components (e.g., polymers, excipients, etc.) for use in controlled-release formulations, and methods of producing the same, are also described, e.g., in U.S. Pat. No. 4,863,742, which is incorporated by reference for these purposes.

The release of the active ingredient can be slowed or controlled by using, for example, hydroxypropylmethyl cellulose in varying proportions to provide the desired release profile, other polymer matrices, gels, permeable membranes, osmotic systems, multilayer coatings, microparticles, liposomes, microspheres, or the like, or combinations thereof. Examples of suitable delayed- or controlled-release formulations are known to those of ordinary skill in the art, and may readily be selected for use with the short elimination half-life cardiovascular drug formulations of the present invention. Thus, tablets, capsules, gelcaps, caplets, and the like, that are adapted for controlled-release, may be used in accordance with the presently disclosed methods. The controlled-release of the active ingredient may be triggered or stimulated by various inducers, for example pH, temperature, enzymes, water, or other physiological conditions or compounds.

The controlled-release formulations used in the present methods may include any number of pharmaceutically acceptable excipients. Suitable excipients include, but are not limited to, carriers, such as sodium citrate or dicalcium phosphate; fillers or extenders, such as stearates, silicas, gypsum, starches, lactose, sucrose, glucose, mannitol, talc, or silicic acid; binders, such as hydroxymethyl-cellulose, alginates, gelatin, polyvinyl pyrrolidone, sucrose or acacia; humectants, such as glycerol; disintegrating agents, such as agar, calcium carbonate, potato or tapioca starch, alginic acid, certain silicates, or sodium carbonate; solution retarding agents, such as paraffin; absorption accelerators, such as quaternary ammonium compounds; wetting agents, such as cetyl alcohol or glycerol monostearate; absorbents, such as kaolin and bentonite clay; lubricants, such as talc, calcium stearate, magnesium stearate, solid polyethylene glycols, and sodium lauryl sulfate; stabilizers, such as fumaric acid; coloring agents; buffering agents; dispersing agents; preservatives; organic acids; and organic bases. The aforementioned excipients are given as examples only and are not meant to include all possible choices. Additionally, many excipients may have more than one role, or be classified in more than one group; the classifications are descriptive only, and not intended to limit any use of a particular excipient.

Examples of suitable organic acids include, but are not limited to, adipic acid, ascorbic acid, citric acid, fumaric acid, malic acid, succinic acid, tartaric acid, and mixtures thereof. Suitable organic bases, include, but are not limited to, sodium citrate, sodium succinate, sodium tartrate, potassium citrate, potassium tartrate, potassium succinate, and mixtures thereof. Suitable diluents include, but are not limited to, lactose, talc, microcrystalline cellulose, sorbitol, mannitol, xylitol, fumed silica, stearic acid, magnesium stearate, sodium stearate, and mixtures thereof.

In one embodiment, the controlled-release formulations of the present invention are provided as multiparticulate formulations. At least one short elimination half-life cardiovascular drug is typically formed into an active core by applying the compound to a nonpareil seed having an average diameter in the range of about 0.4 to about 1.1 mm or about 0.85 to about 1.00 mm. The drug may be applied with or without additional excipients onto the inert cores, and may be sprayed from solution or suspension using a fluidized bed coater (e.g., Wurster coating) or pan coating system. Alternatively, the drug may be applied as a powder onto the inert cores using a binder to bind it to the cores. Active cores may also be formed by extrusion of the core with suitable plasticizers (described below) and any other processing aids as necessary.

The controlled-release formulations of the present invention comprise at least one polymeric material, which may be water-soluble or water-insoluble. Suitable water-soluble polymers include, but are not limited to, polyvinyl alcohol, polyvinylpyrrolidone, methylcellulose, hydroxypropylcellulose, hydroxypropylmethyl cellulose or polyethylene glycol, and/or mixtures thereof.

Suitable water insoluble polymers include, but are not limited to, ethylcellulose, cellulose acetate cellulose propionate, cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), poly (ethylene), poly (ethylene) low density, poly (ethylene) high density, poly (ethylene oxide), poly (ethylene terephthalate), poly (vinyl isobutyl ether), poly (vinyl acetate), poly (vinyl chloride), or polyurethane, and/or mixtures thereof.

EUDRAGIT™ polymers (available from Rohm Pharma) are polymeric lacquer substances based on acrylates and/or methacrylates. A suitable polymer that is freely permeable to the active ingredient and water is EUDRAGIT™ RL. A suitable polymer that is slightly permeable to the active ingredient and water is EUDRAGIT™ RS. Other suitable polymers that are slightly permeable to the active ingredient and water, and exhibit a pH-dependent permeability include, but are not limited to, EUDRAGIT™ L, EUDRAGIT™ S, and EUDRAGIT™ E.

EUDRAGIT™ RL and RS are acrylic resins comprising copolymers of acrylic and methacrylic acid esters with a low content of quaternary ammonium groups. The ammonium groups are present as salts and give rise to the permeability of the lacquer films. EUDRAGIT™ RL and RS are freely permeable (RL) and slightly permeable (RS), respectively, independent of pH. The polymers swell in water and digestive juices, in a pH-independent manner. In the swollen state, they are permeable to water and to dissolved active compounds.

EUDRAGIT™ L is an anionic polymer synthesized from methacrylic acid and methacrylic acid methyl ester. It is insoluble in acids and pure water. It becomes soluble in neutral to weakly alkaline conditions. The permeability of EUDRAGIT™ L is pH dependent. Above pH 5.0, the polymer becomes increasingly permeable.

In one embodiment, the polymeric material comprises methacrylic acid co-polymers, ammonio methacrylate co-polymers, or mixtures thereof. Methacrylic acid co-polymers such as EUDRAGIT™ and EUDRAGIT™ L (Rohm Pharma) are particularly suitable for use in the controlled-release formulations of the present invention. These polymers are gastroresistant and enterosoluble polymers. The polymer films are insoluble in pure water and diluted acids. They dissolve at higher pHs, depending on their content of carboxylic acid. EUDRAGIT™ S and EUDRAGIT™ L can be used as single components in the polymer coating or in combination in any ratio. By using a combination of the polymers, the polymeric material may exhibit a solubility at a pH between the pHs at which EUDRAGIT™ L and EUDRAGIT™ S are separately soluble.

The core may comprise a polymeric material comprising a major proportion (i.e., greater than 50% of the total polymeric content) of one or more pharmaceutically acceptable water-soluble polymers, and optionally a minor proportion (i.e., less than 50% of the total polymeric content) of one or more pharmaceutically acceptable water insoluble polymers.

Alternatively, the core may comprise a polymeric material comprising a major proportion (i.e., greater than 50% of the total polymeric content) of one or more pharmaceutically acceptable water insoluble polymers, and optionally a minor proportion (i.e., less than 50% of the total polymeric content) of one or more pharmaceutically acceptable water-soluble polymers. The formulations may optionally contain a coating membrane partially or completely surrounding the core, comprising a major proportion of one or more pharmaceutically acceptable film-forming, water-insoluble polymers, and optionally a minor proportion of one or more pharmaceutically acceptable film-forming, water-soluble polymers. The water insoluble polymer may form an insoluble matrix having a high or low permeability to the cardiovascular drug(s).

In one embodiment, the polymeric material comprises methacrylic acid co-polymers, ammonio methacrylate co-polymers, or mixtures thereof. Methacrylic acid co-polymers such as EUDRAGIT™ S and EUDRAGIT™ L are particularly suitable for use in the controlled-release formulations of the present invention. These polymers are gastroresistant and enterosoluble polymers. The polymer films are insoluble in pure water and diluted acids. They dissolve at higher pHs, depending on their content of carboxylic acid. EUDRAGIT™ S and EUDRAGIT™ L can be used as single components in the polymer coating or in combination in any ratio. By using a combination of the polymers, the polymeric material may exhibit a solubility at a pH between the pHs at which EUDRAGIT™ L and EUDRAGIT™ S are separately soluble.

Ammonio methacrylate co-polymers such as EUDRAGIT™ RS and EUDRAGIT™ RL are also particularly suitable for use in the controlled-release formulations of the present invention. These polymers are insoluble in pure water, dilute acids, buffer solutions, or digestive fluids over the entire physiological pH range. The polymers swell in water (and digestive fluids independently of pH). In the swollen state they are permeable to water and dissolved actives. The permeability of the polymers depends on the ratio of ethylacrylate (EA), methyl methacrylate (MMA), and trimethylammonioethyl methacrylate chloride (TAMCI) groups in the polymer. Those polymers having EA:MMA:TAMCI ratios of 1:2:0.2 (EUDRAGIT™ RL) are more permeable than those with ratios of 1:2:0.1 (EUDRAGIT™ RS). Polymers of EUDRAGIT™ RL are insoluble polymers of high permeability. Polymers of EUDRAGIT™ RS are insoluble films of low permeability.

The ammonio methacrylate co-polymers may be combined in any desired ratio. For example, the ratio of EUDRAGIT™ RS: EUDRAGIT™ RL (90:10) may be used. The ratios may be adjusted to provide a delay in release of the drug. For example, the ratio of EUDRAGIT™ RS: EUDRAGIT™ RL may be about 100:0 to about 80:20, about 100:0 to about 90:10, or any ratio in between. In such formulations, the less permeable polymer EUDRAGIT™ RS would generally comprise the majority of the polymeric material.

The ammonio methacrylate co-polymers may be combined with the methacrylic acid co-polymers within the polymeric material in order to achieve the desired delay in release of the drug. Ratios of ammonio methacrylate co-polymer (e.g., EUDRAGIT™ RS) to methacrylic acid co-polymer in the range of about 99:1 to about 20:80 may be used. The two types of polymers may also be combined into the same polymeric material, or provided as separate coats that are applied to the core.

In addition to the EUDRAGIT™ polymers described above, a number of other copolymers may be used to create a delay in drug release. These include methacrylate ester co-polymers (e.g., EUDRAGIT™ NE™ 30D). Further information on the EUDRAGIT™ polymers is to be found in “Chemistry and Application Properties of Polymethacrylate Coating Systems,” in Aqueous Polymeric Coatings for Pharmaceutical Dosage Forms, ed. James McGinity, Marcel Dekker Inc., New York, pg 109-114).

The polymeric material typically comprises one or more soluble excipients so as to increase the permeability of the polymeric material. Suitably, the soluble excipient is selected from among a soluble polymer, a surfactant, an alkali metal salt, an organic acid, a sugar, and a sugar alcohol. Such soluble excipients include polyvinyl pyrrolidone, polyethylene glycol, sodium chloride, surfactants such as sodium lauryl sulfate and polysorbates, organic acids such as acetic acid, adipic acid, citric acid, fumaric acid, glutaric acid, malic acid, succinic acid, and tartaric acid and sugars such as dextrose, fructose, glucose, lactose and sucrose, and sugar alcohols such as lactitol, maltitol, mannitol, sorbitol and xylitol, xanthan gum, dextrins, and maltodextrins. In some particular embodiments, polyvinyl pyrrolidone, mannitol, and/or polyethylene glycol are the soluble excipients. The soluble excipient is typically used in an amount of from about 1% to about 10% by weight, based on the total dry weight of the polymer.

The polymeric material can also include one or more auxiliary agents such as a filler, a plasticizer, and/or an anti-foaming agent. Representative fillers include talc, fumed silica, glyceryl monostearate, magnesium stearate, calcium stearate, kaolin, colloidal silica, gypsum, micronized silica, and magnesium trisilicate. The quantity of filler used typically ranges from about 2% to about 300% by weight, and may range from about 20 to about 100%, based on the total dry weight of the polymer. In one embodiment, talc is the filler.

The coatings can also include a material that improves the processing of the polymers. Such materials are generally referred to as plasticizers and include, for example, adipates, azelates, benzoates, citrates, isoebucates, phthalates, sebacates, stearates, and glycols. Representative plasticizers include acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-1-octyl phthalate, di-1-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. In one embodiment, the plasticizer is dibutyl sebacate. The amount of plasticizer used in the polymeric material typically ranges from about 10% to about 50%, for example, about 10, 20, 30, 40, or 50%, based on the weight of the dry polymer.

In one embodiment, the anti-foaming agent is simethicone. The amount of anti-foaming agent used typically comprises from about 0% to about 0.5% of the final formulation.

The amount of polymer to be used in controlled-release formulations is typically adjusted to achieve the desired drug delivery properties, including the amount of drug to be delivered, that rate, timing, and location of drug delivery, the time delay of drug release, and the size of the multiparticulates in the formulation. The amount of polymer applied typically provides about a 10 to about 100% weight gain to the cores. In one embodiment, the weight gain from the polymeric material is about 25 to about 70%.

The combination of all solid components of the polymeric material, including co-polymers, fillers, plasticizers, and optional excipients and processing aids, typically provides about a 10 to about 450% weight gain on the cores. In one embodiment, the weight gain is about 30 to about 160%.

The polymeric material may be applied by any known method, for example, by spraying using a fluidized bed coater (e.g., Wurster coating) or pan coating system.

The coated cores are typically dried or cured after application of the polymeric material. Curing means that the multiparticulates are held at a controlled temperature for a time sufficient to provide stable release rates. Curing may be performed for example in an oven or in a fluid bed drier. Curing may be carried out at any temperature above room temperature.

A sealant or barrier may be applied to the polymeric coating. A sealant or barrier layer may also be applied to the core prior to applying the polymeric material. The sealant or barrier layer does not modify the release of short elimination half-life cardiovascular drug(s) significantly. Suitable sealants or barriers are permeable or soluble agents such as hydroxypropyl methylcellulose, hydroxypropyl cellulose, hydroxypropyl ethylcellulose, and xanthan gum. Hydroxypropyl methylcellulose is particularly useful in this regard.

Other agents may be added to improve the processability of the sealant or barrier layer. Such agents include talc, colloidal silica, polyvinyl alcohol, titanium dioxide, micronized silica, fumed silica, glycerol monostearate, magnesium trisilicate, magnesium stearate, or a mixture thereof. The sealant or barrier layer may be applied from solution (e.g., aqueous) or suspension using any known means, such as a fluidized bed coater (e.g., Wurster coating) or pan coating system. Suitable sealants or barriers include, for example, OPADRY WHITE Y-1-7000 and OPADRY OY/B/28920 WHITE, both of which are available from Colorcon Limited, England.

The invention also provides an oral dosage form containing a multiparticulate cardiovascular drug formulations as hereinabove defined, in the form of caplets, capsules, particles for suspension prior to dosing, sachets, or tablets. When the dosage form is in the form of tablets, the tablets may be disintegrating tablets, fast dissolving tablets, effervescent tablets, fast melt tablets, and/or mini-tablets. The dosage form can be of any shape suitable for oral administration of a drug, such as spheroidal, cube-shaped oval, or ellipsoidal. The dosage forms may be prepared from the multiparticulates in a manner known in the art and may include additional pharmaceutically acceptable excipients, as desired.

The thickness of the polymer in the formulations, the amounts and types of polymers, and the ratio of water-soluble polymers to water-insoluble polymers in the controlled-release formulations are generally selected to achieve a desired release profile of the cardiovascular drug(s). For example, by increasing the amount of water insoluble-polymer relative to the water soluble-polymer, the release of the drug may be delayed or slowed.

The amount of the drug administered, as well as the dose frequency, will vary depending on the particular dosage form used and the route of administration. The amount and frequency of administration will also vary according to the age, body weight, and response of the individual subject. A competent physician can readily determine typical dosing regimens without undue experimentation. It is also noted that the clinician or treating physician will know how and when to interrupt, adjust, or terminate therapy in conjunction with individual subject response.

In general, the total daily dosage for treating, preventing, and/or managing the cardiovascular conditions described herein is from about 0.1 mg to about 10,000 mg of one or more cardiovascular drugs. One of skill in the art is familiar with the recommended starting dosage amounts for any particular drug. In some embodiments, the cardiovascular drug is the beta-blocker metoprolol, which may be provided in an amount from about 1 mg to about 600 mg, or from about 5 mg to about 400 mg, or from about 10 mg to about 400 mg, or from about 12.5 mg to about 400 mg, or from about 25 mg to about 400 mg, or from about 10 mg to about 200 mg, or from about 10 mg to about 100 mg, or any fraction in between. A single dose may be formulated to contain about 5, 10, 12.5, 25, 50, 100, 200, or 400 mg of metoprolol, or any amount in between. In one embodiment, the beta-blocker(s), or pharmaceutically acceptable salts thereof, comprise about 0.5 to about 20%, about 0.5 to about 8%, or about 0.5 to about 4% of the total weight of the formulation.

Any of the pharmaceutical compositions and dosage forms described herein may further comprise one or more additional pharmaceutically active compounds. Such compounds may be included to treat, prevent, and/or manage the same condition being treated, prevented, and/or managed with the drug that is already present, or a different condition altogether. Those of skill in the art are familiar with examples of the techniques for incorporating additional active Ingredients into compositions comprising cardiovascular drugs. Alternatively, such additional pharmaceutical compounds may be provided in a separate formulation and co-administered to a subject with a cardiovascular drug formulation according to the present invention. Such separate formulations may be administered before, after, or simultaneously with the administration of the cardiovascular drug formulations of the present invention. In one embodiment, the cardiovascular formulation is co-administered with one or more other compounds including, but not limited to: beta-blockers; diuretics, in particular, thiazide diuretics (e.g., hydrochlorothiazide); inotropic agents; antiplatelet agents; statins (e.g., atorvastatin, cerivastatin, fluvastatin, lovastatin, pravastatin, resuvastatin, simvastatin); vasodilators (coronary, peripheral, and/or pulmonary); peripheral adrenergic blockers; central adrenergic blockers; mixed alpha/beta adrenergic blockers; angiotensin converting enzymes (ACE) inhibitors; angiotensin II receptor antagonists; antiarrhythmics (groups I, II, and III); calcium channel blockers; and/or nitrates.

The invention is further illustrated by reference to the following examples. It will be apparent to those skilled in the art that many modifications, both to the materials and methods, may be practiced without departing from the purpose and scope of the invention.

### EXAMPLES

### Example 1

Preparation of Chronotherapeutic Metoprolol Formulations

Metoprolol instant-release multiparticulates were prepared as follows:

| | Amount |
|---|---|
| Ingredient | (kg) |
| Metoprolol Tartrate | 40.00 |
| Non Pareil Seeds | 40.00 |
| Klucel | 1.25 |
| Purified Water | 50.00 |
| Purified water (Flush) | |

The Klucel was dissolved in the purified water and then the metoprolol tartrate was slowly added to the solution with stirring. Stirring was continued until all of the metoprolol tartrate was dissolved. The non pariel seeds were placed in a Glatt fluidized coating machine and heated to fluidize the seeds. The metoprolol/Klucel solution was then sprayed on the non pariel seeds until all of the solution had been applied. The spray lines were flushed with 200 g of water and the product was dried for 15 minutes with an inlet temperature of 65° C.

The instant-release multiparticulates produced above are then coated with a polymer system to produce the desired in-vivo profile, as exemplified below.

| | Amount |
|---|---|
| Ingredient | (kg) |
| Metoprolol Instant release | 10.00 |
| multiparticulates | |
| Eudragit ® S 100 | 10.624 |
| Dibutyl Sebecate | 2.131 |
| Talc | 5.320 |
| Isopropyl Alcohol | 146.80 |
| Purified water | 5.099 |
| Isopropyl Alcohol (flush) | 0.500 |
| Total | 28.075 |

The isopropyl alcohol (146.8 kg) and purified water (5.099 kg) were mixed in a stainless steel drum. While mixing continued, 10.624 kg of Eudragit® S100 was added. Mixing was continued until the Eudragit® S100 had dissolved. Dibutyl sebecate (2.131 kg) was added and the solution was mixed for an additional 15 minutes. The talc (5.320 kg) was added and mixed with the other components for 30 minutes to produce the modified-release coating solution. The fluid bed coating machine was heated to an exhaust temperature of 40° C. before the metoprolol instant-release microparticulates (10 kg) were added. The modified-release coating solution was then sprayed onto the metoprolol instant-release microparticulates until the amount required to produce the desired percent potency was applied. The percent potency (100×mg Metoprolol/Total mg weight) of the modified-release multiparticulates varies with the amount of coating solution applied. In vitro release data for a range of different percent potency mutiparticulate batches are shown below:

| | % Released In-Vitro | | | |
|---|---|---|---|---|
| Batch(% Potency) | A(24%) | B(22%) | C(20%) | D(17.5%) |
| Acid 2 Hours | 0 | 0 | 0 | 0 |
| Buffer 1 Hour | 3 | 2 | 1 | 0 |
| Buffer 2 Hour | 10 | 8 | 4 | 2 |
| Buffer 4 Hour | 54 | 40 | 24 | 11 |
| Buffer 6 Hour | 91 | 88 | 74 | 38 |
| Buffer 8 Hour | 94 | 95 | 94 | 79 |
| Buffer 10 Hour | 95 | 95 | 95 | 95 |

### Example 2

Simulations Determining Preferred Pharmacokinetic Profiles Based on T_{Max}, Peak-to-Trough Ratio, and Time of Therapeutic Coverage (50% Cₘₐₓ) for Chronotherapeutic Metoprolol Formulations Having Varying Lag Times

Plasma concentration versus time curves were simulated using WinNonlin Version 4.0.1 based on the equation:
C(t)=D*K01/V/(K01−K10)*(EXP(−K10*t)−EXP(−K01*t))

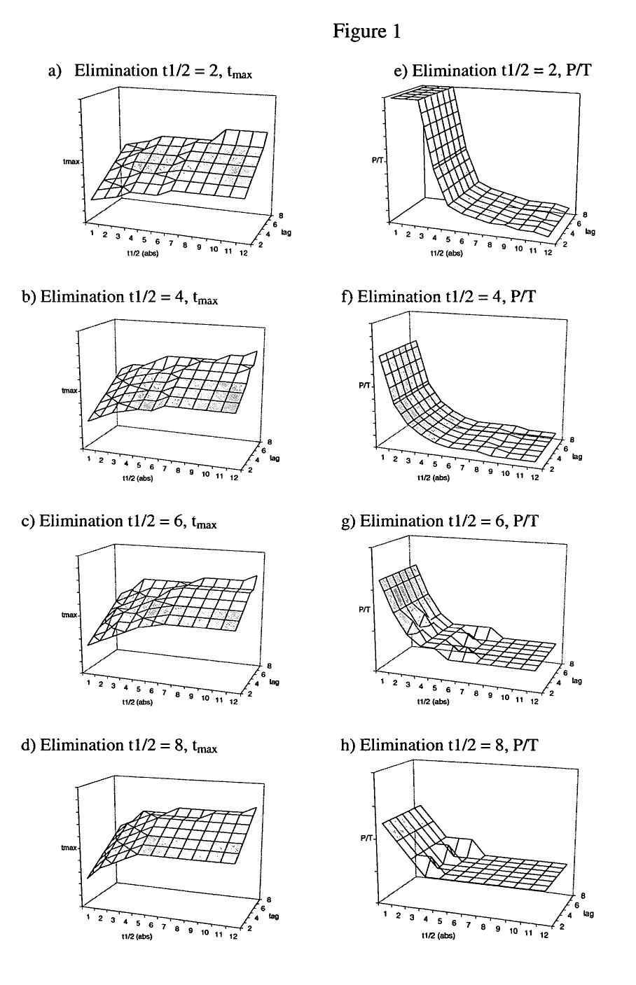

(D=Dose, V=Volume of Distribution, K01=absorption rate constant=ln2/absorption half-life, and K10=elimination rate constant=ln2/elimination half-life). Dose and Volume were chosen arbitrarily and are not used in the subsequent calculations. The data were projected to steady-state with a 24 h dosing interval, using the linear superposition principle (WinNonlin). Tₘₐₓ and Peak/Trough (P/T) were estimated from the steady-state plasma concentration versus time data and time cover at 50% of Cₘₐₓ was estimated for those curves where tₘₐₓ=8-12 h and P/T≧4. FIG. 1 (a)-(d) illustrates the relationship between absorption half-life and lag-time on tₘₐₓ for elimination half-lives of 2, 4, 6, and 8 hours, respectively. FIG. 1 (e)-(h) illustrates the relationship between absorption half life and lag-time on P/T for elimination half-lives of 2, 3, 6, and 8 hours, respectively, where the shaded areas indicate the combinations where tₘₐₓ=8-12 h and P/T≧4. Table 2 summarizes the time cover at 50% Cₘₐₓ for the combinations where tₘₐₓ=8-12 h and P/T≧4.

TABLE 2
Time Cover (h) at 50% Cₘₐₓ, where tₘₐₓ = 8-12h and P/T ≧ 4
(Bold with an * indicates time cover ≧ 12h)
Elimination t1/2 = 2h

| | Absorption t1/2 (h) | | | | | | | | | |
|---|---|---|---|---|---|---|---|---|---|---|
| Lag (h) | 1 | 2 | 3 | 4 | 5 | 6 | 7 | 8 | 9 | 10 |
| 2 | | 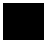 | | | | | | | | |
| 3 | | 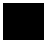 | | | | | 14* | 15* | | |
| 4 | | 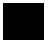 | | 10 | 11 | 13* | 14* | 15* | | |
| 5 | | 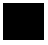 | 9 | 10 | 11 | 13* | 14* | 15* | | |
| 6 | | 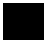 | 8 | 10 | 11 | 13* | 14* | 15* | | |
| 7 | 5 | 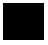 | 8 | 10 | 11 | 13* | 14* | 15* | | |
| 8 | 5 | 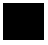 | 8 | 10 | 11 | 13* | 14* | 15* | | |

Elimination t1/2 = 4h

| | Absorption t1/2 (h) | | | | | | | | | |
|---|---|---|---|---|---|---|---|---|---|---|
| Lag (h) | 1 | 2 | 3 | 4 | 5 | 6 | 7 | 8 | 9 | 10 |
| 2 | | | | 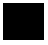 | 16* | | | | | |
| 3 | | | 12* | 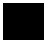 | 16* | | | | | |
| 4 | | 10 | 12* | 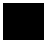 | 16* | | | | | |
| 5 | 8 | 10 | 12* | 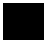 | 16* | | | | | |
| 6 | 8 | 10 | 12* | 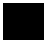 | 16* | | | | | |
| 7 | 8 | 10 | 12* | 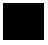 | 16* | | | | | |
| 8 | 8 | 10 | 12* | 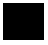 | | | | | | |

Elimination t1/2 = 6h

| | Absorption t1/2 (h) | | | | | | | | | |
|---|---|---|---|---|---|---|---|---|---|---|
| Lag (h) | 1 | 2 | 3 | 4 | 5 | 6 | 7 | 8 | 9 | 10 |
| 2 | | | | | | 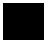 | | | | |
| 3 | | | 15* | | | 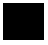 | | | | |
| 4 | | 13* | 15* | | | 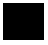 | | | | |
| 5 | 10 | 13* | 15* | | | 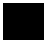 | | | | |
| 6 | 10 | 13* | 15* | | | 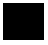 | | | | |
| 7 | 10 | 13* | 15* | | | 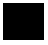 | | | | |
| 8 | 10 | 13* | | | | 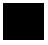 | | | | |

Elimination t1/2 = 8h

| | Absorption t1/2 (h) | | | | | | | | | |
|---|---|---|---|---|---|---|---|---|---|---|
| Lag (h) | 1 | 2 | 3 | 4 | 5 | 6 | 7 | 8 | 9 | 10 |
| 2 | | | | | | | | 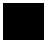 | | |
| 3 | | 15* | | | | | | 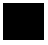 | | |
| 4 | | 15* | | | | | | 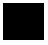 | | |
| 5 | 12* | 15* | | | | | | 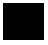 | | |
| 6 | 12* | 15* | | | | | | 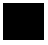 | | |
| 7 | 12* | 15* | | | | | | 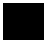 | | |
| 8 | 12* | 15* | | | | | | 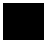 | | |

### Example 3

Comparison of Metoprolol Formulations

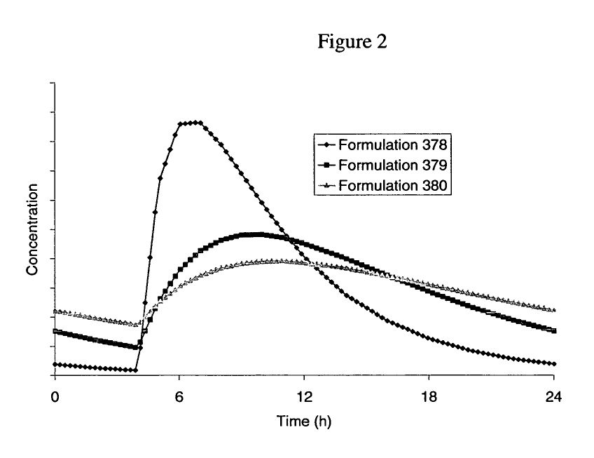

Delayed onset, extended release formulations of metoprolol tartrate were simulated as described above. The elimination half-life of metoprolol is 3.5 hours. A four hour lag was considered appropriate for a simulated metoprolol tartrate formulation. Formulations with absorption half-lives of 1 h (Formulation 378), 5 h (Formulation 379) and 10 h (Formulation 380) were simulated and projected to steady state as described above. FIG. 2 illustrates the steady-state plasma concentration versus time curves for Formulations 378-380.

| | | | | | | | Time Cover |
|---|---|---|---|---|---|---|---|
| Formulation | t½(abs) | | Tₘₐₓ | Cₘₐₓ | Cₘᵢₙ | P/T | (50% Cₘₐₓ) |
| 378 | 1 | hour | 6.79 | 0.86 | 0.02 | 43 | 6.79 |
| 379 | 5 | hours | 9.94 | 0.48 | 0.10 | 5 | 14.55 |
| 380 | 10 | hours | 10.91 | 0.39 | 0.17 | 2 | 21.58 |

Formulation 379 achieved all the desired characteristics of the invention, i.e., time of peak concentrations (Tₘₐₓ) between 8 and 12 hours, peak-to-trough fluctuation (P/T)≧4, and time cover (50% of Cₘₐₓ)≧12 hours. Formulation 378 achieved peak concentrations too early (6.79 h) and only maintained concentrations above 50% of Cₘₐₓ for 6.79 h. Formulation 380 only achieved peak-to-trough fluctuations of 2, while meeting the other criteria.

### Example 4

Use of a Chronotherapeutic Controlled-Release Metoprolol Formulation to Treat a Subject Suffering from Hypertension

A subject who is currently taking a formulation of metoprolol for the management of hypertension is switched to a chronotherapeutic formulation according to the present invention. The formulation is administered at night, prior to bedtime. The delay in onset coupled with the tapering of release at the end of the dosing interval ensures that the subject obtains a therapeutic effect during the morning and throughout the day, but also has a sufficiently long drug free period at the end of the day. The drug free period coincides with the lowest risk period for cardiovascular complications (nighttime and sleeping hours) for the safety and comfort of the subject. The treating physician will recognize the need to modify the dose according to the severity and frequency of symptoms. The recommended starting dose is 50 mg or 100 mg, once-daily. At the judgment of the treating physician the dose may be increased to 400 mg, once daily, after several days.

What is claimed is:

1. A pharmaceutical formulation comprising at least one cardiovascular drug that exhibits an in vivo elimination half-life of less than about 8 hours, wherein the formulation exhibits the following in vivo profile following administration to a subject:
a) a delay in release of therapeutic levels of the at least one drug for about 2 to about 8 hours;
b) a Tₘₐₓ at about 8 to about 12 hours;
c) a drug plasma level within 50% of the peak for greater than or equal to 12 hours; and
d) a peak-to-trough ratio of drug plasma levels greater than or equal to about 4.

2. The formulation of claim 1, wherein the in vivo elimination half-life of the at least one cardiovascular drug is less than about 2, 3, 4, 5, 6, 7, 8, or any fraction in between.

3. The formulation of claim 1, wherein the delay in release of therapeutic concentrations of the cardiovascular drug is about 2, 3, 4, 5, 6, 7, or 8 hours, or any hour or fraction of time in between, following administration to the subject.

4. The formulation of claim 1, wherein the Tₘₐₓ occurs at about 8, 9, 10, 11, or 12 hours, or any hour or fraction of time in between, following administration to the subject.

5. The formulation of claim 1, wherein the drug plasma level is within 50% of the peak for about 12, 13, 14, 14, 16, 17, 18, 19 or 20 hours, or any hour or fraction of time in between, from the time of administration.

6. The formulation of claim 1, wherein the peak-to-trough ratio is about 4:1, 5:1, 6:1, 7:1, 8:1, 9:1, or 10:1, or any whole number or fraction in between.

7. The formulation of claim 1, wherein the cardiovascular drug is selected from among peripheral alpha or beta blockers, central alpha or beta blockers, mixed alpha/beta blockers, angiotensin converting enzymes (ACE) inhibitors, angiotensin II receptor antagonists, antiarrhythmics (groups I, II, or III), calcium channel blockers, potassium channel activators, aldosterone antagonists, renin inhibitors, diuretics, and coronary, peripheral, and pulmonary vasodilators.

8. The formulation of claim 1, wherein the cardiovascular drug is metoprolol.

9. The formulation of claim 1, wherein the cardiovascular drug is the tartrate salt of metoprolol.

10. The formulation of claim 1, wherein the cardiovascular drug is Nicorandil.

11. The formulation of claim 1, wherein the formulation further comprises one or more additional cardiovascular drugs.

12. The formulation of claim 1, wherein the formulation is coated with one or more polymers chosen from water-soluble polymers, water-insoluble polymers, and combinations thereof.

13. The formulation of claim 12, wherein the polymer is chosen from polyvinyl alcohol, polyvinylpyrrolidone, methyl cellulose, hydroxypropyl cellulose, hydroxypropyl methyl cellulose, polyethylene glycol, ethylcellulose, cellulose acetate, cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene), poly(propylene), poly(ethylene oxide), poly(ethylene terephthalate), poly(vinyl isobutyl ether), poly(vinyl acetate), poly(vinyl chloride), polyurethane, and mixtures thereof.

14. A method of treating one or more cardiovascular conditions comprising administering, to a subject in need of such a treatment, a pharmaceutical formulation comprising at least one cardiovascular drug that exhibits an in vivo elimination half-life of less than about 8 hours; wherein the formulation exhibits the following in vivo profile following administration to a subject:
a) a delay in release of therapeutic levels of the at least one drug for about 2 to about 8 hours;
b) a Tₘₐₓ at about 8 to about 12 hours;
c) a drug plasma level within 50% of the peak for greater than or equal to 12 hours; and
d) a peak-to-trough ratio of drug plasma levels greater than or equal to about 4.

15. The method of claim 14, wherein the pharmaceutical formulation is administered one time per day.

16. The method of claim 14, wherein the cardiovascular condition is chosen from among hypertension, angina, coronary artery disease, cerebrovascular disease, peripheral vascular disease, myocardial infarction, stroke, congestive heart failure, angina pectoris, hypertension, and thrombosis.

17. The method of claim 14, wherein the in vivo elimination half-life of the at least one cardiovascular drug is less than about 2, 3, 4, 5, 6, 7, 8, or any fraction in between.

18. The method of claim 14, wherein the delay in release of therapeutic concentrations of the cardiovascular drug is about 2, 3, 4, 5, 6, 7, or 8 hours, or any hour or fraction of time in between, following administration to the subject.

19. The method of claim 14, wherein the Tₘₐₓ occurs at about 8, 9, 10, 11, or 12 hours, or any hour or fraction of time in between, following administration to the subject.

20. The method of claim 14, wherein the drug plasma level is within 50% of the peak for about 12, 13, 14, 14, 16, 17, 18, 19 or 20 hours, or any hour or fraction of time in between, following administration to the subject.

21. The method of claim 14, wherein the peak-to-trough ratio is about 4:1, 5:1, 6:1, 7:1, 8:1, 9:1, or 10:1, or any whole number or fraction in between.

22. The method of claim 14, wherein the cardiovascular drug is selected from among peripheral alpha or beta blockers, central alpha or beta blockers, mixed alpha/beta blockers, angiotensin converting enzymes (ACE) inhibitors, angiotensin II receptor antagonists, antiarrhythmics (groups I, II, or III), calcium channel blockers, potassium channel activators, aldosterone antagonists, renin inhibitors, diuretics, and coronary, peripheral, and pulmonary vasodilators.

23. The method of claim 14, wherein the cardiovascular drug is metoprolol.

24. The method of claim 14, wherein the cardiovascular drug is the tartrate salt of metoprolol.

25. The method of claim 14, wherein the amount of metoprolol administered is from about 1 mg to about 600 mg per day.

26. The method of claim 14, wherein the amount of metoprolol administered is from about 10 mg to about 400 mg per day.

27. The method of claim 14, wherein the cardiovascular drug is Nicorandil.

28. The method of claim 14, wherein the formulation further comprises one or more additional cardiovascular drugs.

29. The method of claim 14, wherein the formulation is coated with one or more polymers chosen from water-soluble polymers, water-insoluble polymers, and combinations thereof.

30. The method of claim 29, wherein the water soluble polymer is chosen from polyvinyl alcohol, polyvinylpyrrolidone, methyl cellulose, hydroxypropyl cellulose, hydroxypropyl methyl cellulose, polyethylene glycol, ethylcellulose, cellulose acetate, cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene), poly(propylene), poly(ethylene oxide), poly(ethylene terephthalate), poly(vinyl isobutyl ether), poly(vinyl acetate), poly(vinyl chloride), polyurethane, and mixtures thereof.

31. The method of claim 14, wherein the pharmaceutical formulation further comprises one or more additional pharmaceutically active compounds.

32. The method of claim 15, wherein the cardiovascular formulation is administered at night.

33. A method of reducing the effects of the rebound phenomena in a subject that is to be withdrawn from a cardiovascular drug comprising replacing the cardiovascular drug being administered to the subject with a formulation according to claim 1 that contains the cardiovascular drug to be withdrawn, and administering that formulation for at least about 7 days before ceasing the administration of the cardiovascular drug.

34. A method of preventing long-term desensitization to a cardiovascular drug therapy in a subject comprising administering a formulation according to claim 1 to the subject in need of such prevention.

35. The formulation of claim 8, further comprising a statin drug.

36. The formulation of claim 35, wherein the statin drug is atorvastatin, cerivastatin, fluvastatin, lovastatin, pravastatin, resuvastatin, or simvastatin.

37. The formulation of claim 11, wherein at least one of the one or more additional cardiovascular drugs is a statin drug.

38. The formulation of claim 37, wherein the statin drug is atorvastatin, cerivastatin, fluvastatin, lovastatin, pravastatin, resuvastatin, or simvastatin.

39. The method of claim 23, wherein the pharmaceutical formulation further comprises a statin drug.

40. The method of claim 39, wherein the statin drug is atorvastatin, cerivastatin, fluvastatin, lovastatin, pravastatin, resuvastatin, or simvastatin.

41. The method of claim 31, wherein at least one of the one or more additional cardiovascular drugs is a statin drug.

42. The method of claim 41, wherein the statin drug is atorvastatin, cerivastatin, fluvastatin, lovastatin, pravastatin, resuvastatin, or simvastatin.
